(19) United States
(12) Patent Application Publication
(10) Pub. No.: US 2013/0274693 A1
(43) Pub. Date: Oct. 17, 2013

(54) Zero-G Liquid Dispenser

(71) Applicants: Mark R. NOWAKOWSKI, San Diego, CA (US); Vito Frank ASARO, San Diego, CA (US); Craig R. NOWAKOWSKI, Columbus, CA (US)

(72) Inventors: Mark R. NOWAKOWSKI, San Diego, CA (US); Vito Frank ASARO, San Diego, CA (US); Craig R. NOWAKOWSKI, Columbus, CA (US)

(21) Appl. No.: 13/650,106

(22) Filed: Oct. 11, 2012

Related U.S. Application Data
(60) Provisional application No. 61/546,027, filed on Oct. 11, 2011.

(51) Int. Cl. A61F 9/00

(52) U.S. Cl. CPC A61F 9/0008
USPC 604/298

(57) ABSTRACT

Improved liquid dispenser devices configured to deliver liquid compositions to a user's eye are described, as well as methods for making and using such devices.

### RELATED APPLICATION

This application claims the benefit of, and priority to, U.S. provisional patent application Ser. No. 61/546,027, filed 11 Oct. 2011, the contents of which are hereby incorporated by reference in their entirety for any and all purposes.

### BACKGROUND OF THE INVENTION

1. Field of the Invention

The present invention concerns improved liquid dispensers, particularly dispensers for delivering liquids and solutions to any eye of a patient.

The following description includes information that may be useful in understanding the present invention. It is not an admission that any of the information provided herein, or any publication specifically or implicitly referenced herein, is prior art, or even particularly relevant, to the presently claimed invention.

2. Background

A number of eyedropper designs are known. Generally, eyedroppers include a nozzle having an opening in communication with a flexible bulb that acts as a fluid reservoir. Typically, the flexible bulb contains an eye treatment liquid such that the eyedropper may be inverted such that the nozzle opening is positioned below the flexible bulb, which allows solution from the flexible bulb to flow into the nozzle by gravity. In this orientation, slight pressure applied to the flexible bulb discharges a drop of the eye treatment solution into the user's eye.

There are several factors that often complicate the conventional way of instilling eye drops using conventional eyedroppers. For example, popular and well-known eyedroppers are supplied for delivery of optical solutions such as Visine™ and Clear Eyes™. In these well-known configurations, to use the eyedropper a user must tilt back her/his head to a horizontal or near-horizontal orientation in order to introduce the eye treatment solution to the eye to be treated. Tilting one's head in this way is difficult for some individuals, especially the elderly, to elevate the shoulder high enough to place the eyedropper in an ideal position above the eye. Limitation of motion of the hand or the wrist can also make it difficult to turn the bottle in a substantially inverted position. Tilting the head back can also be distracting and potentially dangerous in certain situations, for example, while driving an automobile or for persons having trouble maintaining their balance. Additionally, the gravity-induced free fall of the drop(s) can be difficult to control, resulting in drops partially or completely missing the target eye and instead hitting the user's face or other surface. The user thus wastes the medication being dispensed. Moreover, a child user may be unwilling or unable to use a conventional eyedropper properly or at all. Additionally, if a user fails to accurately place a complete drop into the eye, or places too many drops into the eye, the intended benefits of the medicated liquid may be diminished or lost.

As is also known, with conventional eyedroppers the internal pressure of the fluid reservoir decreases as the solution is discharged, and the internal pressure continues to become lower than the atmospheric pressure after each dose. To address this, conventional eyedroppers are designed to permit the in-flow of ambient air through the nozzle opening in order to equalize the pressure imbalance inside and outside of the device to equalize. Of course, the in-flowing ambient air may contaminated with microbes, particulates, etc. Since a variety of microbes can be introduced into the solution in the eyedropper, certain preservatives are often added to the solution to assure sterility. However, as is widely known in the art, preservatives, especially in large doses, themselves often have harmful side effects.

Conventional eyedroppers are also known to be difficult to position and stabilize when introducing a solution to the eye. For example, an apparatus using a nasal bridge piece as a support (see, e.g., U.S. Pat. No. 4,257,417) or an apparatus using a nasal bridge piece and two additional facial points-of-contact pieces (see, e.g., U.S. patent application publication no. 2011/0098664 A1) requires the user to at least rest a nasal bridge piece to on the bridge of a user's nose. As a result, such devices require users to repeatedly contact their faces with potentially unclean surfaces; moreover, such devices are cumbersome and difficult to transport.

The instant invention not only addresses these shortcomings, it also provides safer and more functionally reliable devices that preclude reflexive blinking before a drop makes contact with the eye. As is known, reflexive blinking is influenced by visual clues and tactile sensation. If an object suddenly moves toward the eye, the eyelids reflexively close at high speed and the head flinches—a reflex to a visual threat without voluntary control. On the other hand, if a blast of air hits the eye, the eye will reflexively blink even though it cannot “see” the air coming; instead, the cornea “feels” the air because of tactile sensation. Various devices have been developed to address this problem. For example, a device has been developed that has a ring-shaped base that a user must fit in the orbital of the eye to engage the eyelids (see, e.g., U.S. Pat. No. 5,810,794). The eyelids are retracted and preclude reflexive blinking. As with other conventional eyedroppers, however, such a device requires the user to repeatedly contact the face with potentially unclean surfaces, and the device is also cumbersome and difficult to transport.

Devices are also known that introduce a predetermined amount or dosage of eye treatment solution to a user's eye. See, e.g., U.S. patent application publication no. 2004/0039355 A1. However, costly electronics and other components, the need for a power source, and the lack of portability precludes the use of such a device in many applications.

In recent years, the use of packaging containers made of a plastic has increased dramatically. With the increasing use of disposable plastic containers, including conventional plastic eyedrop containers, the public hazard caused by discarded plastic containers and the effective utilization of resources have become increasingly important issues to address. Indeed, today many municipalities and other government agencies are beginning to require a more ecologically sound approach to packaging consumer products, including such as eyedroppers, for example, by requiring manufacturers that utilize plastic packaging to recover plastic containers after use or by drastically reducing the amount of plastics used in packaging.

This invention addresses these and other shortcomings of conventional eyedroppers and like devices, thereby permitting more precise dosing and simple, single-handed operation without the need for the user to tilt her/his head back, in particular for administering eye drops.

### SUMMARY OF THE INVENTION

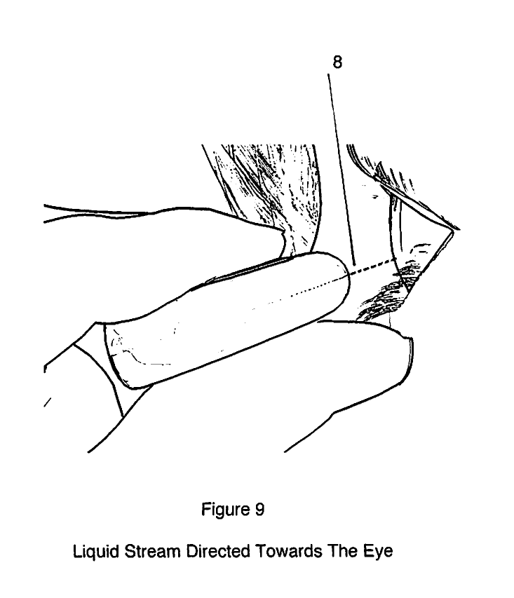

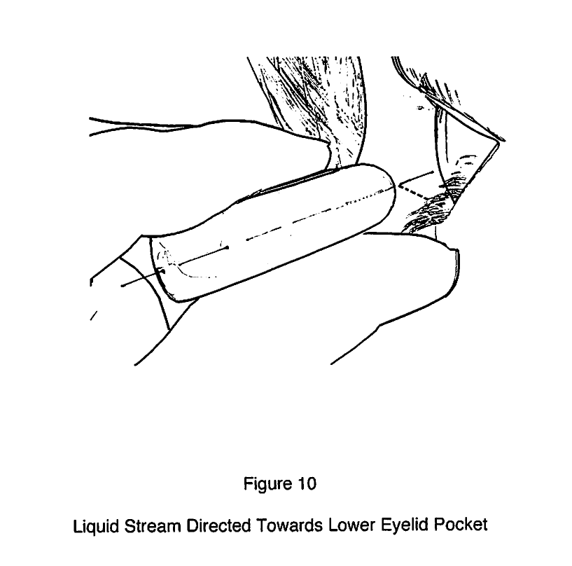

This object is achieved, according to a first aspect of the invention, by a novel, inventive, and useful disposable liquid dispenser having a collapsible liquid reservoir provided for single-handed operation, the liquid reservoir having at least one dosing opening and being bounded at least in some sections by flexible wall sections. “Single-handed operability” of a dispenser means such that the dispenser is typically held between the thumb and a finger, preferably the index finger, of the same hand and can be compressed by exertion of an actuating force. The liquid stored in the liquid reservoir is therefore pressurized and can be discharged through a nozzle having one or more nozzle orifices (i.e., a dosing opening) assigned to the liquid reservoir. Preferably, the reservoir or dispenser is ergonomically designed such that the distal end of the user's thumb contacts the lower eye lid (or skin on the face just below the lower eyelid), allowing the retraction of the lower eye lid, and a stabilizing element is provided to stabilize and position the device for controlled, accurate dispensing of the liquid directly into the eye, as shown in FIG. 9 or the pocket formed between the eye and the lower eye lid, as shown in FIG. 10.

### BRIEF DESCRIPTION OF THE DRAWINGS

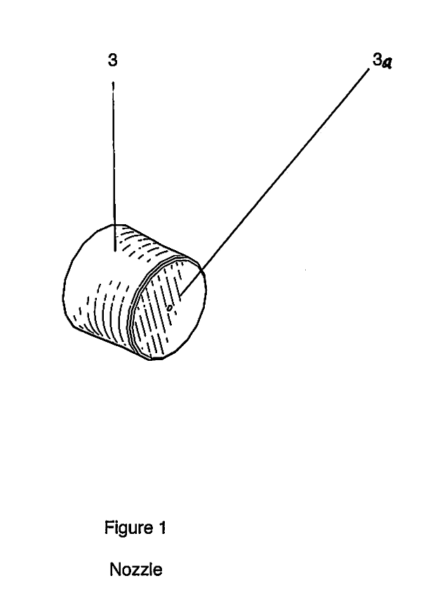

The above and other aspects, features, and advantages of the present invention will be more apparent from the following more particular description thereof, presented in conjunction with the drawings in FIGS. 1-12.

Corresponding reference characters indicate corresponding components throughout the several views of the drawings. Skilled artisans will appreciate that elements in the figures are illustrated for simplicity and clarity and have not necessarily been drawn to scale. For example, the dimensions, sizing, and/or relative placement of some of the elements in the figures may be exaggerated relative to other elements to help to improve understanding of various embodiments. Also, common but well-understood elements that are useful or necessary in a commercially feasible embodiment are often not depicted in order to facilitate a less obstructed view of these various embodiments. It will also be understood that the terms and expressions used herein have the ordinary meaning as is usually accorded to such terms and expressions by those skilled in the corresponding respective areas of inquiry and study except where other specific meanings have otherwise been set forth herein.

### DETAILED DESCRIPTION OF THE INVENTION

The following description is not to be taken in a limiting sense, but is made merely for the purpose of describing the general principles of the embodiments described herein. The scope of the invention should be determined with reference to the claims. The present embodiments address the problems described in the background while also addressing other additional problems as will be seen from the following detailed description.

Any suitable self-sealing, flexible material that can be adapted for the production of nozzles, such as shown in FIG. 1, can be employed. Generally, a nozzle (3) is constructed of a resiliently flexible material such as natural rubber. By squeezing the reservoir to increase its internal pressure, the flexible nozzle associated with an opening or port in the reservoir expands to make a deformation, which in turn causes the nozzle orifice(s) (3a) to take an outward open position, allowing the dispensing of a controlled liquid stream or series of liquid droplets to escape from the nozzle orifice (or orifices if multiple orifices are provided in the particular nozzle), independent of the position of the device or gravity. Dispensing of liquid from the device ceases once the actuating force experienced by the collapsible reservoir is removed and the reservoir internal pressure is balanced with atmospheric pressure. In this way, the orifice(s) in the self-sealing, flexible nozzle (3) acts as a one-way valve device, assuring the sterility of the liquid remaining in the collapsible reservoir, and in which it is also ensured that no contents of the container independently exit from the reservoir.

Such a self-sealing nozzle can be fabricated from any suitable material, or combination or materials, utilizing various methods, including injection molding, compression molding, casting, or other methods. Typically, the nozzle is composed of a sufficiently pliable biocompatible material, typically having a durometer of between 0 and about 50, and preferably approved for use by the FDA for such applications, and characterized with the desired self-sealing properties. It has been shown that the type of material, wall thickness at the distal end of the nozzle, and diameter of the orifice(s) through the wall together play a role in determining the pressure required to expand an orifice sufficiently to allow fluid to flow from the reservoir. An orifice can be formed during the fabrication stage of the nozzle or may be formed after the fabrication stage by using a needle, laser, or other object or device to form the orifice.

The collapsible reservoir can be fabricated using a number of different biocompatible, flexible materials, including polymers, foils, waterproof papers, or other materials (or combinations or layers of such materials) capable of containing aqueous solutions. The aforementioned material(s) can be modified by methods including blow molding, injection molding, heat sealing, or other fabrication methods resulting in the formation of a liquid compartment that can accommodate the liquid for long periods of time without leaking or degrading. In addition, the resulting reservoir must be able to withstand compression by exertion, often repeated exertions, of an actuating force.

Some embodiments of the invention employ a dual-chambered collapsible reservoir that includes a first chamber capable of containing aqueous solutions positioned in functional association with a second chamber designed to contain a gas, for example, air. Preferably, when external pressure is not being applied to the reservoir, the pressure of the gas in the second chamber corresponds to atmospheric pressure.

An integral part of a collapsible reservoir of a device according to the invention is a liquid dispensing portion or tube compromising a proximal end and a distal end. The tube may be part of the reservoir or may be affixed to the reservoir separately. The internal pressure of liquid reservoir is therefore increased and the liquid contained in the reservoir can be discharged through the orifice(s) in the nozzle assigned to the liquid reservoir.

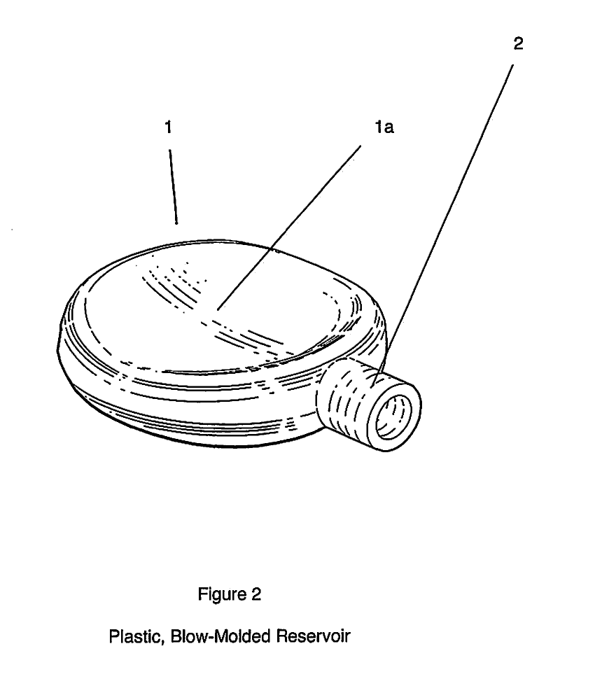

A representative example of such a collapsible reservoir is illustrated in FIG. 2. In this example, the collapsible reservoir (1) is composed of a flexible polymer, such as polypropylene, which in the depicted embodiment can be produced by blow molding or another suitable technique. The resulting reservoir is comprised of a hollow container (1a) and an integrated liquid dispensing tube (2). Alternatively, separate mating halves (or a greater number of sub-units) of the reservoir can be formed, for example, by injection molding, and permanently attached using, for example, an adhesive, heating sealing technique, or similar type of method for forming a water-tight seal between the mating halves. Preferably, production results in a reservoir in which the container (1a) and liquid dispensing tube (2) are a unitary component.

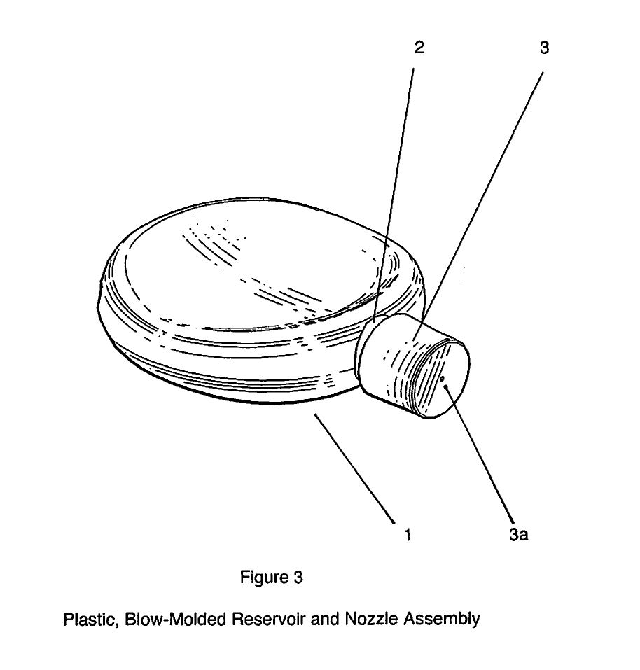

The desired fluid is aseptically added to the collapsible reservoir (1) and the nozzle (3) is subsequently assembled to the reservoir liquid dispensing tube (2) as shown in FIG. 3. In a preferred method of fabrication, the nozzle (3) is permanently bonded to the reservoir dispensing tube (2) by sealing, for example, using an adhesive, ultrasonic welding, RF welding, or other suitable method.

In general the nozzle (3) and collapsible reservoir (1) of the present invention can be manufactured using conventional methods of aseptic manufacturing. This aseptic manufacturing process refers to manufacturing and packaging of sterile liquids, wherein the formation of the nozzle and reservoir, filling the reservoir with liquid, such as the desired ophthalmic fluids, and formation of the seal to the container is achieved aseptically, all in a clean and controlled environment.

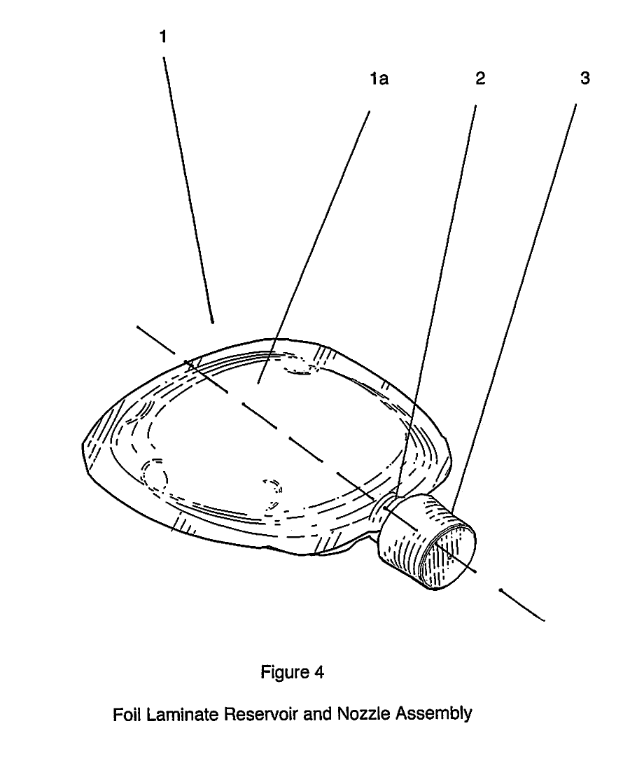

Shown in FIG. 4 is an alternative reservoir (1) fabricated using two pieces of die cut foil laminate and a liquid dispensing tube (2). The liquid dispensing tube is preferentially formed in a separation operation, by one of many fabrication methods, such as the extrusion of polyethylene. The three pieces are subsequently assembled and sealed by RF welding or other joining methods known to those familiar with such techniques. The desired fluid is aseptically added to the reservoir (1) and the nozzle (3) is subsequently assembled to the reservoir liquid dispensing tube (2). In a preferred method of fabrication, the nozzle (3) is permanently bonded to the reservoir dispensing tube (2) by sealing with an adhesive, ultrasonic welding, RF welding, or other method. The central axis of the reservoir is shown by the hatched line extending through the device.

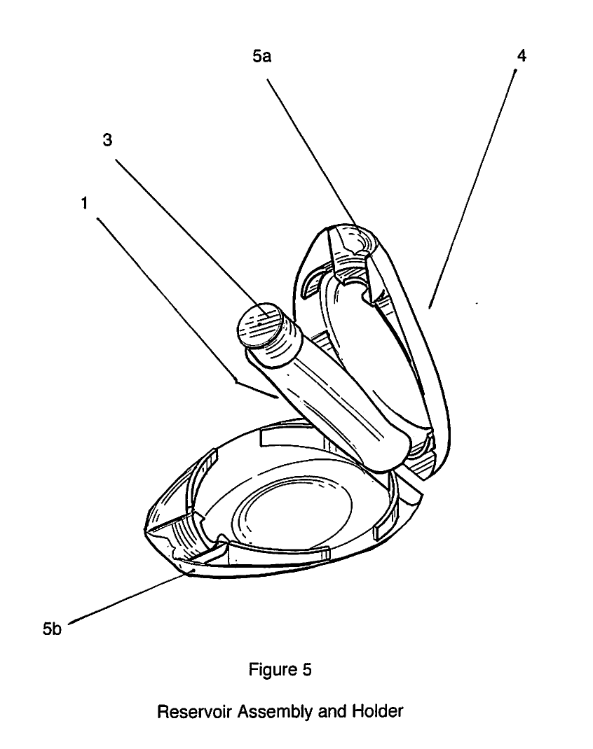

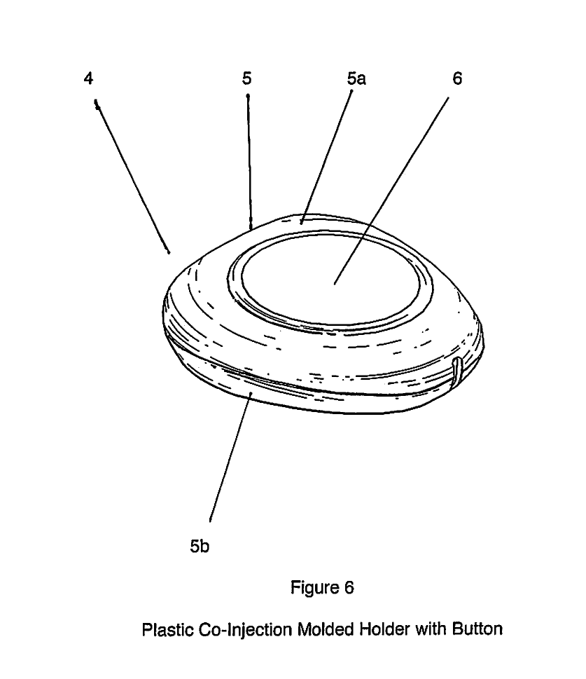

In an alternative embodiment, such as shown in FIG. 5, a reservoir assembly, consisting of a collapsible reservoir (1) fabricated by one of the methods previously described, a nozzle (3), and the desired liquid, is inserted into a reusable, injection molded holder or housing (4), which in the depicted embodiment is made from two hinged holder component halves (5a, 5b). A preferred holder can be co-injection molded as shown in FIG. 6. In such a design, the holder or housing (4) includes a rigid plastic body (5) made from two hinged halves (5a, 5b) and a pliable, plastic button (6), which is actuated by, for example, a user's index finger, which compresses the reservoir and causes discharging of a portion of the liquid (preferably an aqueous solution intended for ocular delivery) through the nozzle (not shown). If desired, the housing can also include a hinged or removable rigid or semi-rigid cap or cover (not shown) that closes over the button (6) to as to prevent it from being inadvertently depressed, for example, when being inadvertently contacted while a person rummages around in her purse, for example.

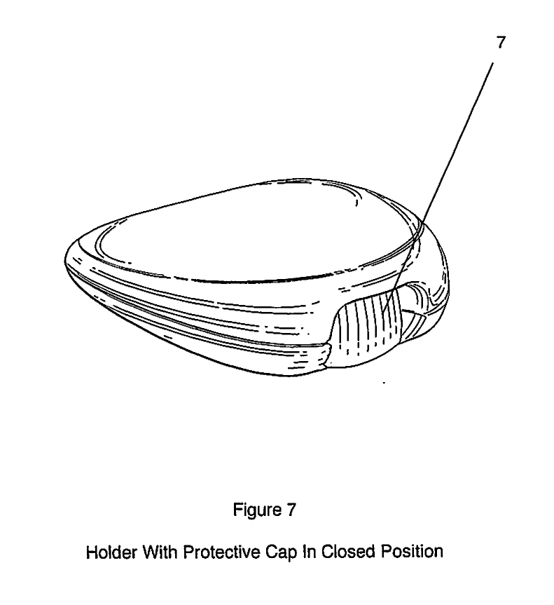

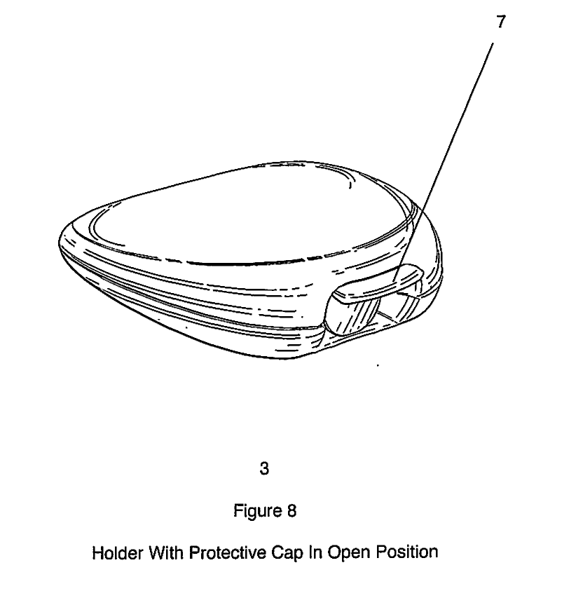

There are many advantages in utilizing such holder or housing (4), including a reduction of material used in the fabrication of the reservoir assembly, a design that is ergonomically superior to a stand-alone reservoir assembly, and an optional protective cap to reduce contamination of the nozzle. FIG. 7 shows an embodiment having a protective cap (7) in the closed position, while FIG. 8 shows such an embodiment having the protective cap (7) in the open position, exposing the nozzle (3).

FIG. 9 shows an illustration of a person using a liquid dispensing device according to the invention, for example, a liquid dispenser as shown in any of FIGS. 1-8, to dispense a fine, pressurized stream of liquid (hatched line, 8) from the collapsible reservoir along the central axis (20) of the device onto the surface of the person's eye without the need for the person to tilt her/his head back. In this embodiment, the liquid expelled from the device traverses along the central axis if the dispenser. For example, when using a liquid dispenser as shown in FIG. 5 or 6, the ergonomically shaped liquid dispenser allows the user to position his/her index finger on the button (6) disposed in the upper portion (5a) of the housing (4) while at the same time using the thumb of the same hand to both support the liquid dispenser and also steady and space the liquid dispenser a suitable distance (e.g., from about 0.1 cm to about 4 cm, preferably from about 0.25 cm to about 2.5 cm) from the user's eye.

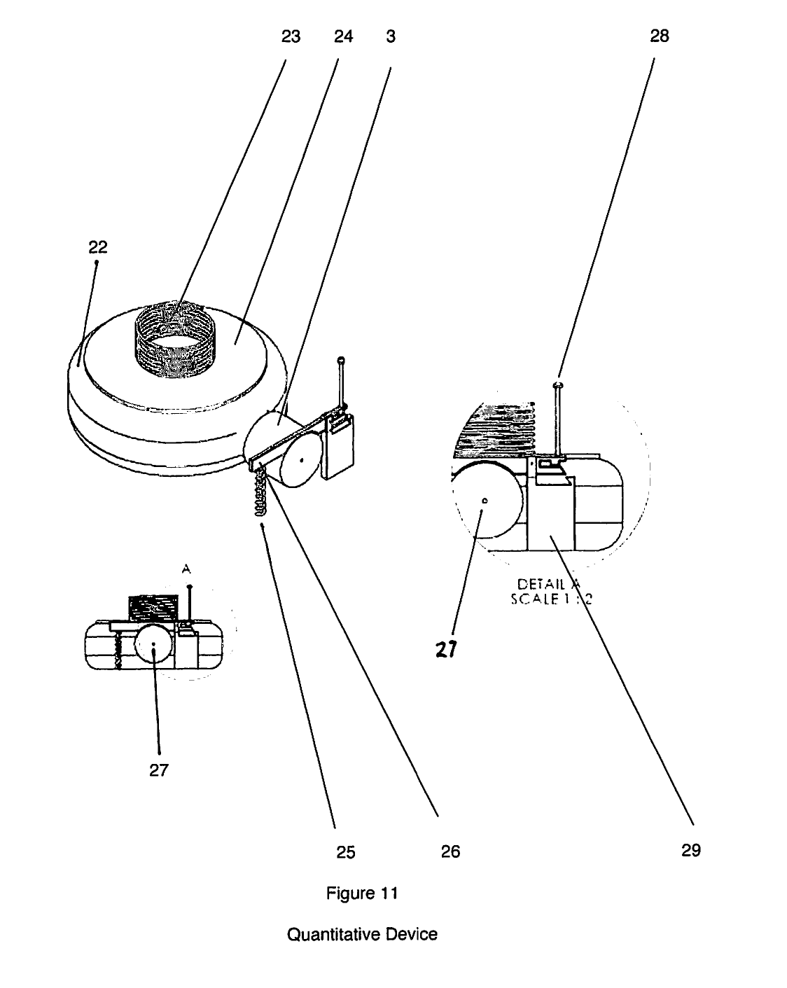

FIG. 10 also shows an illustration of a person using a liquid dispensing device according to the invention, for example, a liquid dispenser as shown in any of FIGS. 1-8, to dispense a fine, pressurized stream of liquid (hatched line, 8) from the reservoir of the device into a pocket (9) created by the user gently pulling her/his lower eyelid slightly downward using her/his thumb. At the same time as the user creates the pocket, s/he holds the liquid dispenser in the same hand and, using, for example, the index finger of that hand, depresses the button (6) to cause a fine, pressurized stream of liquid (hatched line, 8) to be delivered into the pocket (9). After dispensing the solution, the user releases her/his thumb from her/his face, closing the pocket (9). Here, the user again uses a liquid dispenser as shown in FIG. 5 or 6. Such an ergonomically shaped liquid dispenser allows the user to position his/her index finger on the button (6) disposed in the upper portion (5a) of the housing (4) while at the same time using the thumb of the same hand to both steady and support the liquid dispenser and also create a lower eyelid pocket. Preferably, the user positions the liquid dispenser a suitable distance (e.g., from about 0.1 cm to about 4 cm, preferably from about 0.25 cm to about 2.5 cm) from her/his eye before dispensing liquid from the dispenser. As illustrated in FIG. 10, the liquid flowing out of the dispenser flows along a path that is at an angle from the central axis of the dispenser. The angle is determined by configuration of the orifice(s) in the nozzle.

Whereas conventional eyedrop dispensers discharge a non-specific volume of fluid, the embodiments of a dispenser according to the invention represented in FIG. 11 introduce a predetermined amount or dosage of eye treatment solution to the eye, for example, from about 1-250 uL or more of solution, including about 5 uL, 10 uL, 25 uL, 50 uL, and 100 uL. In these embodiments, a constant, compressive force is applied to the exterior of the collapsible reservoir while maintaining an independent secondary force to the perimeter of the nozzle. The pressure applied to the collapsible reservoir and the amount of time that the force is removed from the nozzle determines the amount of fluid that will be dispensed. For example, if 50 g of compressive force is applied to the exterior of the reservoir and the force normally applied to the perimeter of the nozzle is removed for 0.5 seconds, 25 uL of solution (e.g., deionized water, an allergy-relieving solution, an ocular medicine, etc.) can be accurately and precisely discharged.

As shown in FIG. 11, a constant compressive force is applied to the exterior of the collapsible reservoir (22) via a spring (23) under compression that bears on a plate (24) disposed between the collapsible reservoir (22) and spring (23). As those in the art will understand, the force applied to the exterior of reservoir (22) may be the result of any type of compression, cantilever, or other type of spring capable of storing energy when compressed, such as the compression spring (23). The spring can be fabricated from steel, plastic, or any other type of material known by persons with knowledge in the art.

An independent secondary force is applied to the perimeter of the nozzle (3) by a coil spring (25) (or other biasing member) and a pressure arm (26) resulting in engaging the nozzle orifice and closing the nozzle orifice in fluid communication with the reservoir (22). In these embodiments, the nozzle orifice (27) is normally closed until the user sufficiently reduces or removes the secondary force to a portion of the nozzle perimeter by pressing a button (28), which opens the nozzle orifice (27) and allows fluid to be expelled from the reservoir (22). The amount of time that the nozzle remains open is determined by a controller (29). The result is the dispensing of a predetermined amount of liquid from the reservoir due to the internal pressure being greater than ambient pressure as a result of the force applied to the exterior of the reservoir.

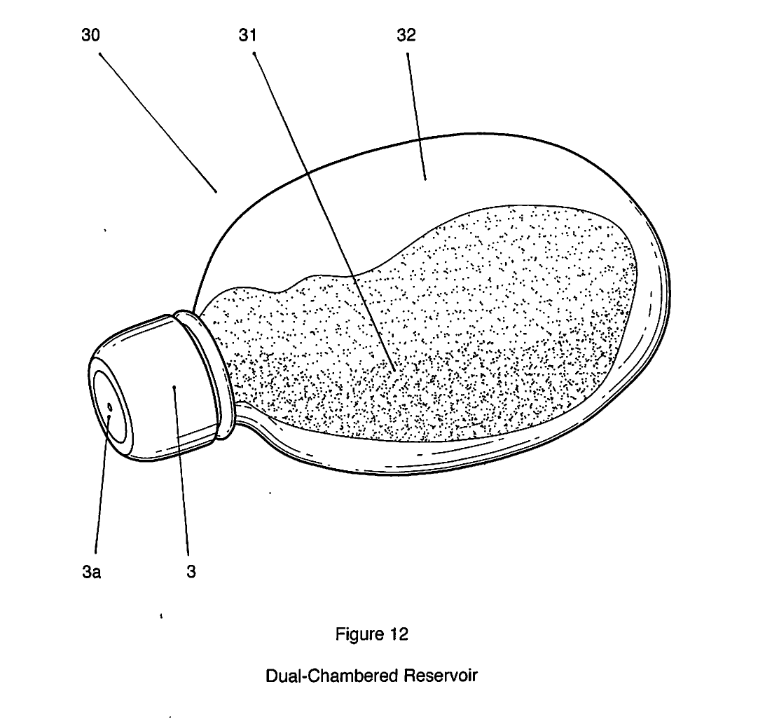

FIG. 12 shows an alternative configuration of a collapsible reservoir that can be used in the invention, namely one having multiple chambers, namely one having a dual-chambered reservoir (30) having first and second chambers (31, 32). The first chamber (31) is designed to contain the fluid (e.g., an aqueous solution) for ocular delivery, while the second chamber (32) contains a gas, for example, air. The first chamber (31) is connected to a liquid dispensing tube, with which a nozzle (3) is associated. The second chamber (32) is functionally associated with the first chamber (31), and preferably envelops, the first chamber (31). In some embodiments, when external pressure is not being applied to the reservoir, the pressure of the gas in the second chamber (32) corresponds to atmospheric pressure. In such embodiments, the second chamber preferably contains a valve that allows pressure inside the second chamber to slowly equalize with the atmospheric pressure of the surrounding environment after an actuating force has been applied thereto. In other embodiments, the second chamber may be pressurized and then sealed so that a pressure above atmospheric pressure is applied to the surface of the first chamber. In any event, when a sufficient actuating force (i.e., the force necessary to overcome the cracking pressure necessary to force open the orifice(s) in the nozzle to allow liquid to be expelled from the fluid-containing chamber/reservoir) is applied to the reservoir (30), directly or indirectly (e.g., as can occur when such a reservoir (30) is substituted for the reservoir (1) within the housing (4) of the dispenser represented in FIG. 5), increased pressure in the second chamber (32) increases pressure on the liquid in the first chamber (31), which causes liquid to be expelled through an orifice (3a) in the nozzle (3).

Dispensing liquid into a user's eye is a common way to deliver medicine and/or solutions to the eye. The normal tear film over an eye consists of three layers: an outer lipid or oily layer, a middle aqueous or watery layer, and an inner mucin layer that holds the rest of the tear film to the cornea and outer structures of the eye. Tear volume in a normal, healthy eye is estimated to be about six microliters, yet conventional eye droppers typically deliver from about 30 to about 60 uL, or from 5-10 times of an eye's normal tear volume. One of the advantages afforded by the instant invention is the ability to deliver far smaller amounts of solution to an eye, for example, from about 1-30 uL, 1-20 uL, or 1-10 uL, particularly about 1 uL, 2 uL, 3 uL, 4 uL, 5 uL, 6 uL, 7 uL, 8 uL, 9 uL, 10 uL, 11 uL, 12 uL, 13 uL, 14 uL, or 15 uL. Depending on such factors as the size, shape, and number of orifices in the nozzle of a dispenser according to the invention, liquid dispensed from the collapsible reservoir may be in the form of small, preferably as a stream or as small, preferably consistently sized, drops.

The delivery of s fine stream of solution or very small droplets is important in the treatment of many ocular conditions, particularly those involving or “dry eye” syndrome (also known as keratitis sicca, keratoconjunctivitis sicca, or xerophthalmia), as millions of people suffer from some type of tear dysfunction. Many individuals do not make an adequate amount of tears and thus the eye may have symptoms of burning, irritation or sandy feeling, itching, and even a decrease in visual acuity since the tear film is responsible for maintaining good vision. Instilling large volumes into the eyes of such people using conventional eye drop technology may be harmful, particularly if large volumes of solution irrigate away the mucin, lipid layer, and proteins normally present in tear film. As will be appreciated, instilling smaller solution volumes may allow a user to instill solution more frequently while still preserving beneficial components of the tear film that the eye or surrounding tissues naturally produces. More frequent and/or smaller volumes may also help repair dehydrated cells of the cornea and conjunctiva, along with providing more comfort to the eye. Smaller instilled volumes will also require less need for removing excess solution from the eye and/or eyelids, such as by wiping with a tissue.

Contact lens wearers also often have symptoms of eye dryness, especially when lenses are worn for long periods of time or in conditions where dehydration of the eye occurs. As wearers of contact lens are aware, when large volumes of solutions are applied to eyes to combat dryness, a contact lens can “float” and slide off the cornea. Accordingly, using a dispenser according to the invention to deliver smaller volumes of hydrating solutions to the eyes of contact lens wearers will still provide relief from contact lens-related symptoms while reducing the likelihood of causing lenses to move out of place. More frequent administration will also be possible, without the difficulties that come from excessive moisture.

There are numerous other applications for dispensers according to the invention, as well. For example, many ocular diseases and conditions are treated by liquid compositions that comprise one or more active pharmaceutical ingredients in a suitable liquid, often aqueous, carrier. Diagnostic uses are also envisioned. Better ocular delivery will allow for improved therapeutic outcomes and a reduction of side effects, as well as reducing cost associated with wasting medicines that simply wash of an eye because too much volume has been delivered. Diagnostic applications are also envisioned. For example, applanation tonometry is commonly used to test intraocular pressure for glaucoma testing. For such testing, a drop of about 50 uL containing sodium fluorscein and a topical anesthetic is typically used. Much smaller volumes of the test reagents, however, can be used, resulting in cost savings as well as reducing the amount of excess dye in the eye and on the eyelids and allowing for better biomicroscope resolution of abnormal stain patterns that could be concealed by larger fluid volumes.

While the present invention has been described by means of specific embodiments and applications thereof, other modifications, variations, and arrangements of the present invention may be made in accordance with the above teachings other than as specifically described to practice the invention within the spirit and scope defined by the following claims.

What is claimed:

1. A liquid dispenser, comprising:
a. a collapsible liquid reservoir comprised of a flexible, biocompatible material, optionally a foil or plastic, to form the reservoir and having a liquid dispensing tube comprising a proximal end and a distal end, wherein the reservoir contains a liquid composition; and
b. a nozzle comprised of a biocompatible elastomer that includes at least one pressure activated self-sealing liquid pore, wherein the nozzle is configured for sealing association with the distal end of the liquid dispensing tube,
wherein the liquid dispenser optionally further comprises at least one of the following:
c. a rigid housing that comprises (i) a reservoir chamber for housing the collapsible liquid reservoir, wherein the reservoir chamber comprises an aperture that allows access to and pressurization of the collapsible liquid reservoir, and (ii) a nozzle port aligned with the pressure activated self-sealing liquid pore(s) of the nozzle; and
d. a cap or seal, which can be operated by one hand, protecting the pressure activated self-sealing liquid pore prior to first use.

2. A liquid dispenser according to claim 1 configured to dispense a predetermined volume of liquid from the collapsible liquid reservoir.

3. A method of delivering a liquid to an eye, comprising using a liquid dispenser according to claim 1 to dispense liquid from the reservoir to an eye of a user, thereby delivering a liquid to the eye of the user.
